(19) United States
(12) Patent Application Publication
(10) Pub. No.: US 2011/0257484 A1
(43) Pub. Date: Oct. 20, 2011

(54) LIGHT SOURCE DEVICE AND ENDOSCOPE APPARATUS USING THE SAME

(75) Inventors: Akira MIZUYOSHI, Kanagawa (JP); Kazuhiko NAKAMURA, Kanagawa (JP)

(73) Assignee: FUJIFILM CORPORATION, Tokyo (JP)

(21) Appl. No.: 13/086,284

(22) Filed: Apr. 13, 2011

(30) Foreign Application Priority Data
Apr. 15, 2010 (JP) P2010-094347

(51) Int. Cl. A61B 1/06; G02B 6/04; H01L 33/02; G02B 6/00

(52) U.S. Cl. 600/178; 362/551; 362/554; 362/555

(57) ABSTRACT

A light source device includes: a light emission part in which a plurality of illuminants are arranged on a support body; a light guide member in which light emitted from the light emission part is introduced into an incidence surface at one end of the light guide member; and a light collecting member which is located between the light emission part and the light guide member. The light collecting member includes a plurality of tapered columnar bodies, which are placed so that tip portions thereof are opposed to the incidence surface of the light guide member and base end portions thereof are opposed to light emission surfaces of the illuminants. A selective translucent member for limiting transmission of an infrared component is located along an optical path leading from the light emission part to the incidence surface of the light guide member.

The present application priority from Japanese Patent Application No. 2010-094347 filed on Apr. 15, 2010, the entire content of which is incorporated herein by reference.

### BACKGROUND OF INVENTION

1. Field of the Invention

The present invention relates to a light source device and an endoscope apparatus using the same.

2. Description of the Related Art

Generally, as an illumination light source for a medical or industrial endoscope apparatus, a xenon lamp has been widely used, but nowadays a power-saving and small-size light emitting diode (LED), having a long replacement cycle for a light source, is receiving attention as a light emitting element that takes the place of a xenon lamp. For example, as illustrated in FIG. 18, there is proposed an endoscope apparatus in which a plurality of LEDs 1 are arranged on a support body 2 and through which light emitted from the respective. LEDs 1 is collected via a lens 3 so as to be introduced into an optical fiber bundle of a light guide LG (see JP-A-2000-66115).

However, when light emitted from a light source device side of the apparatus is introduced into an end face of the light guide LG at an endoscope probe side of the apparatus, the light might leak to, for example, an end face of a metal sleeve covering an outer periphery of the optical fiber bundle due to an aberration of the lens 3. In the light guide LG, since necessary light amount varies depending on the type of an endoscope probe, a bundle diameter also varies; hence, the diameter of the metal sleeve also varies for each type of the endoscope probe. In a peroral endoscope apparatus or lower digestive system endoscope, for example, the light guide LG having a large diameter as illustrated in FIG. 19A is provided; on the other hand, in a nasotracheal endoscope or bronchoscope, for example, the light guide LG having a small diameter as illustrated in FIG. 19B is provided. Accordingly, when the light guides LG having different diameters are connected to light source devices via connectors, the smaller the diameter of the light guide LG, the more likely it is that leaked light will be applied to a metal sleeve 4. Further, light emitted from the LED 1 located at an outer edge side of the support body 2, in particular, is likely to be applied to the metal sleeve 4.

Upon application of light to the metal sleeve 4 as mentioned above, light reflected from the end face of the metal sleeve 4 is returned to the light source side to cause temperature increases in the LEDs 1 and the support body 2 on which the LEDs 1 are implemented, thereby reducing luminous efficiency and lifetime of each LED 1. Furthermore, the temperature of an area of the metal sleeve 4 where light is applied will be increased, and thus an adhesive through which fiber bundles are adhered to each other might be degraded by heat.

### SUMMARY OF INVENTION

The present invention has been made in view of the above-described circumstances, and its object is to provide: a light source device that prevents, when light emitted from a light emission part is introduced into a light guide member, a temperature rise in the light emission part, caused by a temperature rise in the periphery of the light guide member and return of reflection light to the light emission part, and that achieves high brightness illumination light with high efficiency; and an endoscope apparatus using such a light source device.

According to a first aspect of the invention, a light source device comprising: a light emission part in which a plurality of illuminants are arranged on a support body; a light guide member in which light emitted from the light emission part is introduced into an incidence surface at one end of the light guide member and through which illumination light is emitted from an emission surface at the other end of the light guide member; and a light collecting member which is located between the light emission part and the light guide member and through which the light emitted from the light emission part is collected into the incidence surface of the light guide member, wherein the light collecting member includes a plurality of tapered columnar bodies tapered toward the light guide member from the light emission part, wherein the plurality of tapered columnar bodies are placed so that tip portions thereof are opposed to the incidence surface of the light guide member, and base end portions thereof are opposed to light emission surfaces of the illuminants, and wherein a selective translucent member that limits transmission of an infrared component, the selective translucent member being located at a portion along an optical path leading from the light emission part to the incidence surface of the light guide member.

A light source device according to the present invention and an endoscope apparatus using the light source device are capable of reliably preventing, when light emitted from a light emission part is introduced into a light guide member, a temperature rise in the light emission part, caused by a temperature rise in the periphery of the light guide member and return of reflection light to the light emission part, thus making it possible to achieve high brightness illumination light with high efficiency.

### BRIEF DESCRIPTION OF THE DRAWINGS

FIG. 1 is a conceptual block diagram of an endoscope apparatus for describing an embodiment of the present invention;

FIG. 2 is an exemplary external view of the endoscope apparatus illustrated in FIG. 1;

FIG. 3 is a schematic diagram of a lighting device;

FIG. 4 is an explanatory diagram illustrating how light is collected by a single tapered columnar body;

FIG. 5A is a schematic diagram illustrating exemplary placement of a light collecting member in which white LEDs are arranged in a 4 by 4 matrix on a support body, base end portions of tapered columnar bodies are opposed to light emission surfaces of the respective white LEDs, and tip portions of the tapered columnar bodies are bundled to provide a light emission window;

FIG. 5B is a partially enlarged diagram illustrating the position of the light emission window in an enlarged manner;

FIG. 6 is a schematic diagram illustrating a case where a concave support body is used;

FIG. 7 is a schematic diagram illustrating a case where an auxiliary illuminant, from which light is emitted toward the light emission window, is placed between tapered columnar bodies which are adjacent to each other;

FIG. 8 is a schematic cross-sectional view of a light emission part in which a fluorescent layer is formed over a support body on which LEDs are implemented;

FIG. 9 is a schematic diagram illustrating a case where a fluorescent material is dispersed in a tapered columnar body;

FIG. 10 is a graph illustrating a spectrum of a combination of light emitted from the LEDs and light emitted from the fluorescent material, and a spectrum of a combination of laser light and light emitted from the fluorescent material;

FIG. 11A is a schematic diagram illustrating an example in which an infrared absorber is provided as the light emission window of the light collecting member including a plurality of tapered columnar bodies;

FIG. 11B is a schematic diagram illustrating an example in which a stub having a multilayer reflection film for selectively reflecting an infrared component is provided at the light emission window of the light collecting member;

FIG. 11C is a schematic diagram illustrating an example in which a stub having an infrared reflection function is provided instead of a dichroic prism of FIG. 11B;

FIG. 12A is an explanatory diagram schematically illustrating how light is introduced into a light guide when a light guide LG having a large diameter is connected to a light source device;

FIG. 12B is a plan view illustrating the lit LEDs on the support body illustrated in FIG. 12A;

FIG. 13A is an explanatory diagram schematically illustrating how light is introduced into a light guide when a light guide LG having a small diameter is connected to the light source device;

FIG. 13B is a plan view illustrating the lit LEDs on the support body illustrated in FIG. 13A;

FIG. 14 is a circuit diagram illustrating a connection circuit of the light emission part in a simplified manner;

FIGS. 15A, 15B, 15C and 15D are explanatory diagrams each schematically illustrating an example of an emitted light pattern in the light emission window;

FIG. 16 is a schematic diagram illustrating a tip portion of an endoscope and a connector;

FIG. 17 is a schematic cross-sectional view illustrating a state in which skew processing has been performed on branched optical fibers;

FIG. 18 is an explanatory diagram illustrating a connection structure between a related light source device and an endoscope probe;

FIG. 19A is an explanatory diagram illustrating a connection state of a related large diameter light guide; and

FIG. 19B is an explanatory diagram illustrating a connection state of a related small diameter light guide.

### DESCRIPTION OF EXEMPLARY EMBODIMENTS

Hereinafter, an embodiment of the present invention will be described in detail with reference to the drawings.

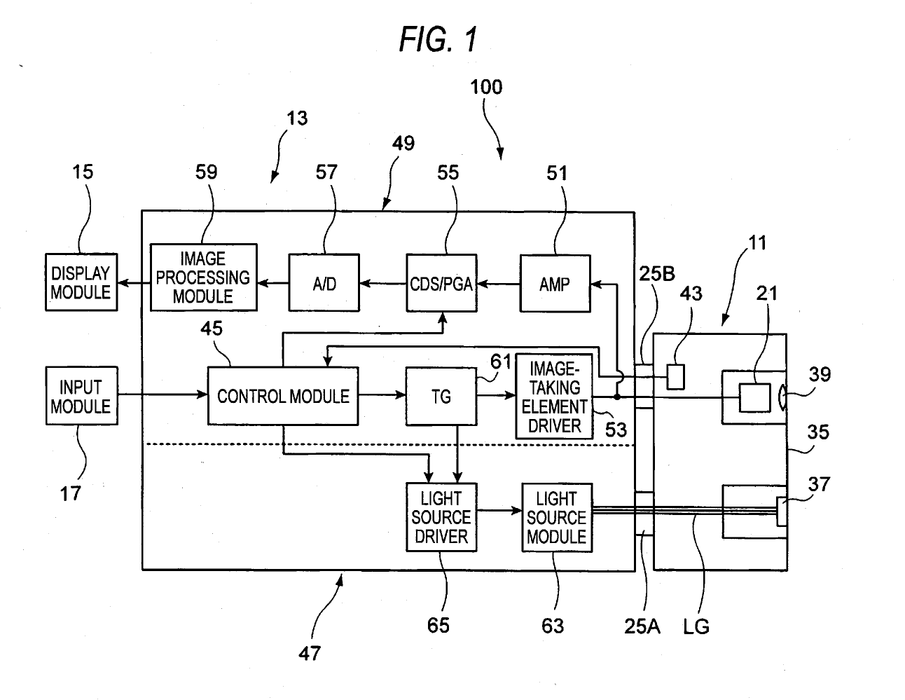

FIG. 1 is a conceptual block diagram of an endoscope apparatus 100 for describing the embodiment of the present invention.

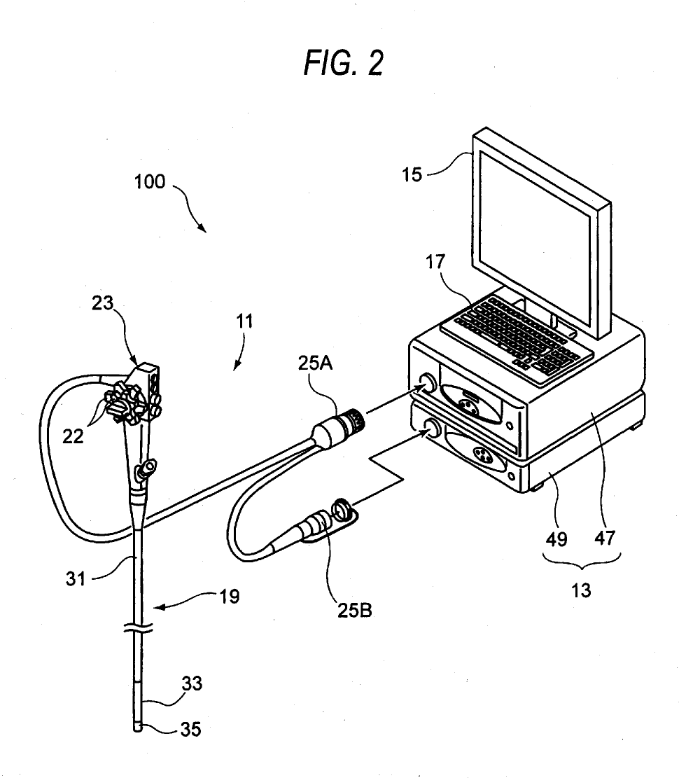

FIG. 2 is an exemplary external view of the endoscope apparatus 100 illustrated in FIG. 1.

As illustrated in FIGS. 1 and 2, the endoscope apparatus 100 includes an endoscope 11; and a controller 13 to be connected with the endoscope 11. The controller 13 is connected with: a display module 15 for displaying information such as image information; and an input module 17 for receiving an input operation. The endoscope 11 is an electronic endoscope including: an illumination optical system for emitting illumination light from a tip of an endoscope insertion part 19 (see FIG. 2) which is to be inserted into a test object; and an image-taking optical system including an image-taking element 21 (see FIG. 1) for taking an image of an observed region.

The endoscope 11 further includes: the endoscope insertion part 19; an operation module 23 (see FIG. 2) through which an operation for bending the tip of the endoscope insertion part 19 and an operation for observation are performed; and connectors 25A and 25B through which the endoscope 11 is detachably connected to the controller 13. Note that although not illustrated, the operation module 23 and the endoscope insertion part 19 are internally provided with various channels such as: a forceps channel through which a tissue collection tool or the like is inserted; an air supply channel; and a water supply channel.

As illustrated in FIG. 2, the endoscope insertion part 19 includes: a soft portion 31 having flexibility; a bendable portion 33; and a tip portion (hereinafter also referred to as an “endoscope tip portion”) 35. As illustrated in FIG. 1, the endoscope tip portion 35 is provided with: an application port 37 through which light is applied to an observed region; and the image-taking element 21 such as a CCD (Charge Coupled Device) image sensor or CMOS (Complementary Metal-Oxide Semiconductor) image sensor for obtaining image information on the observed region. Furthermore, the image-taking element 21 is provided at its light receiving surface with an objective lens unit 39 for forming an observed image.

The bendable portion 33 illustrated in FIG. 2 is allowed to bend by a rotational operation performed on an angle knob 41 located in the operation module 23. The bendable portion 33 may be bent at any angle in any direction in accordance with, for example, a region of a test object for which the endoscope 11 is used, thus allowing the application port 37 and the image-taking element 21 of the endoscope tip portion 35, by which the test object is observed, to be aimed at a desired observed region.

Moreover, different types of the endoscopes 11, such as a nasotracheal endoscope, a peroral endoscope, a lower digestive system endoscope and a bronchoscope, are prepared in accordance with application purposes in a medical field. An endoscope operator attaches the appropriate endoscope to the controller 13 based on a given endoscope examination order, for example. Each endoscope 11 includes a memory (individual information retaining means) 43 for storing various pieces of individual information concerning the type of the endoscope, spectral sensitivity characteristic of the image-taking element, and illumination light. The controller 13 reads the individual information of the connected endoscope 11 from the memory 43, identifies the type of the endoscope 11 by a control module 45, and controls each component so that operative procedure and display are carried out under suitable conditions.

The controller 13 includes: a light source device 47 for producing illumination light to be supplied to the application port 37 of the endoscope tip portion 35; and a processor 49 for performing image processing on an image signal supplied from the image-taking element 21, and the controller 13 is connected to the endoscope 11 via the connectors 25A and 25B. Furthermore, based on instructions provided from the operation module 23 of the endoscope 11 and the input module 17, the processor 49 performs image processing on an image-taking signal transmitted from the endoscope 11, generates an image for display, and supplies the generated image to the display module 15.

The image-taking element 21 is connected with an amplifier (hereinafter abbreviated as “AMP”) 51 and an image-taking element driver 53 which are provided in the processor 49. With a given gain, the AMP 51 amplifies the image-taking signal outputted from the image-taking element 21, and outputs the resulting signal to a correlation double sampling/programmable gain amplifier (hereinafter abbreviated as “CDS/PGA”) 55.

The CDS/PGA 55 outputs the image-taking signal, which has been outputted from the AMP 51, as R, G and B image data corresponding to charge storage amounts of respective light receiving cells of the image-taking element 21, amplifies these pieces of image data, and outputs the resulting image data to an A/D converter 57. The A/D converter 57 converts the analog image data, which has been outputted from the CDS/PGA 55, into digital image data. An image processing module 59 performs various types of image processing on the image data digitized by the A/D converter 57, and outputs an observed image in a body cavity to the display module 15.

The image-taking element driver 53 is connected with a timing generator (hereinafter abbreviated as “TG”) 61 controlled by the control module 45. Using a timing signal (clock pulse) fed from the TG 61, the image-taking element driver 53 controls timing of reading of the image-taking signal (charge storage amount) of the image-taking element 21, shutter speed of an electronic shutter of the image-taking element 21, etc.

The light source device 47 is equipped with components such as: a light source module 63 for supplying illumination light to the application port 37 of the endoscope 11; and a light source driver 65 for controlling the amount of light emitted from the light source module 63. Through a light guide LG including a large number of optical fiber bundles, the light emitted from the light source module 63 is applied to the observed region via the application port 37. Note that in the example of the present embodiment, a plurality of LEDs (light emitting diodes), each having a center emission wavelength of 450 nm to 470 nm, for example, are used as light emitting elements of the light source module 63, and a fluorescent layer containing a fluorescent material excited by blue light emitted from the LEDs is located at light emission surfaces of the LEDs.

The fluorescent material includes a plurality of types of fluorescent substances (e.g., fluorescent materials such as YAG fluorescent material or BAM [BaMgAl₁₀O₁₇]) which absorb part of light emitted from the LEDs and are excited to emit green to yellow light. Thus, the green to yellow light, emitted by excitation using blue light as excitation light, is combined with the light emitted from the LEDs and transmitted through the fluorescent material without being absorbed, thereby producing white (pseudo-white) illumination light. The produced white illumination light will be guided through the light guide LG and applied to the observed region from the application port 37.

As used herein, the “white light” is not strictly limited to light containing all wavelength components of visible light, but may include light having specific wavelength ranges of colors such as R (red), G (green) and B (blue), for example, which are standard colors. For instance, in a broad sense, the “white light” also includes light containing wavelength components of green to red colors, and light containing wavelength components of blue to green colors.

In consideration of a refractive index difference between the fluorescent substances included in the fluorescent material and fixation/solidification resin serving as a filler, the foregoing fluorescent material may be formed of a material in which particle sizes of each fluorescent substance itself and the filler are set so that absorption of infrared light is reduced and dispersion thereof is increased. Thus, for red light or infrared light, dispersion effect is enhanced without reduction in light intensity, and optical loss may be reduced.

Further, the light source driver 65 is connected with the control module 45 and the TG 61. The light source driver 65 supplies a pulse driving current responsive to control carried out by the control module 45 within an exposure period specified by a read pulse responsible for timing of reading of an image-taking signal (stored charge) of the state image-taking element 21, and an electronic shutter pulse, which are provided from the TG 61. In other words, the light source driver 65 is capable of allowing optional illumination light to be applied to the observed region in synchronization with image-taking timing of the image-taking element 21.

As described above, the white light, produced by light emitted from each LED (also referred to as a “white LED”) and light emitted from the fluorescent material by excitation, is applied to the observed region from the tip portion 35 of the endoscope 11. Furthermore, an image of the test object is formed on the image-taking element 21 via the objective lens unit 39, thereby obtaining, as the taken image, the state of the observed region to which illumination light is applied.

The image signal of the taken image, outputted from the image-taking element 21 after the taking of the image, is subjected to signal processing as mentioned above and fed to the image processing module 59. The image processing module 59 performs various types of processing, such as white balance correction, gamma correction, edge enhancement and color correction, on the image-taking signal supplied from the image-taking element 21 and converted into a digital signal, so that together with various pieces of information, the resulting signal is converted into an endoscope observation image and outputted to the display module 15. Moreover, when necessary, the endoscope observation image is stored in an unillustrated storage consisting of a memory or a storage device.

Next, the light source device of the endoscope apparatus 100 with the above-described structure will be described in detail.

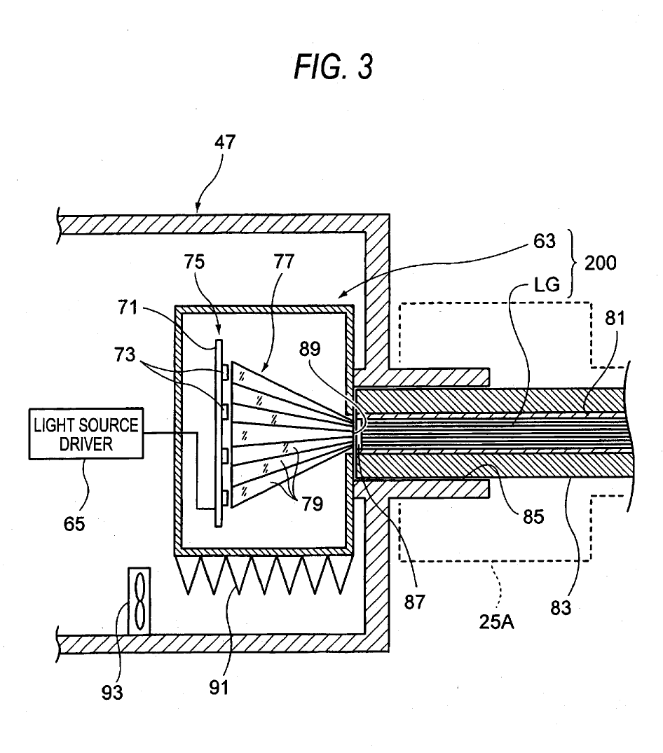

FIG. 3 provides a schematic diagram of a lighting device 200.

The lighting device 200 includes: the light source module 63 incorporated into the above-described light source device 47; and the light guide LG serving as a light guide member, which is connected at one end thereof to a light emission port of the light source module 63 and through which illumination light is emitted from the other end thereof. The light source module 63 includes: a light emission part 75 in which a plurality of white LEDs (illuminants) 73 are arranged on a support body 71 so as to emit light upon reception of supply of power from the light source driver 65; and a light collecting member 77 located between the light emission part 75 and the one end of the light guide LG so as to collect light, emitted from the light emission part 75, into a light incidence surface of the light guide LG.

The light guide LG is the elongated light guide member including: a large number of optical fiber bundles; and a sleeve 81 covering outer peripheries of the optical fiber bundles. The connector 25A is connected to the light source device 47; thus, a protection pipe 83 covering an outer periphery of the sleeve 81 is inserted into an engagement hole 85 while being guided therethrough, and the light guide LG is fixed in a state where a glass window 87 located at a tip of the light guide LG is opposed to a light emission window 89 of the light source module 63.

The sleeve 81 has a cylindrical shape. Examples of materials usable for the sleeve 81 include: metals such as stainless steel and copper alloy; ceramics; crystallized glass; and resin. In particular, zirconia ceramics (zirconium oxide: ZrO₂) may be used since it has translucency for light. By forming the sleeve 81 using zirconia ceramics, a light application range may be increased because even if high intensity light is applied to an end face of the sleeve, the applied light penetrates from the end face of the sleeve into the inside thereof. As a result, a local temperature increase at the end face of the sleeve may be prevented.

Note that the light source module 63 is provided with a heat sink 91, thus allowing heat generated by the light source module 63 to escape outside by air blown by a fan 93.

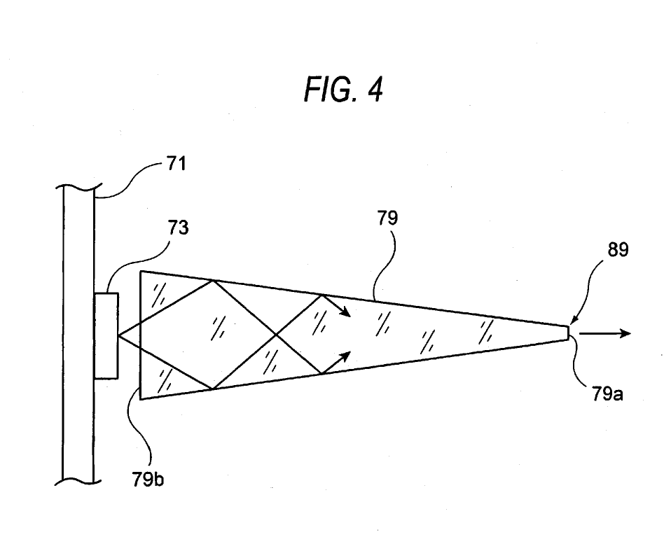

The light collecting member 77 is a collection of a plurality of tapered columnar bodies 79 tapered toward the light guide LG, and the tapered columnar bodies 79 are arranged so that each tapered columnar body 79 is associated with one of the white LEDs 73. FIG. 4 illustrates how light is collected by the single tapered columnar body 79. The tapered columnar body 79 is made of translucent glass or resin, and is a wedge-shaped columnar body that is reduced in cross section toward the front of an optical path. In this embodiment, the tapered columnar bodies 79 each have a triangular shape by which a plurality of the tapered columnar bodies 79 may be bundled at a higher density, but each tapered columnar body 79 may alternatively have a cylindrical shape, other polygonal columnar shape, a conical shape, or a polygonal conical shape.

Each tapered columnar body 79 is placed so that a tip portion 79a of the tapered columnar body 79 is connected to the planar light emission window 89 opposed to the light incidence surface of the light guide LG, and a base end portion 79b of the tapered columnar body 79 is opposed to the light emission surface of the associated white LED 73. Further, the light emitted from the white LED 73 is collected and guided to the tip portion 79a while total reflection is repeated within the tapered columnar body 79. As a result, most of the light emitted from the illuminant is allowed to be incident on the light guide LG as effective light, thus enabling an improvement in light utilization efficiency.

Furthermore, in the tapered columnar body 79, a selective translucent member for limiting transmission of an infrared component is located somewhere along the optical path of at least the tip portion 79a or the base end portion 79b. As this selective translucent member, an infrared cut filter serving as an infrared absorber, for example, may be utilized. Alternatively, the entire tapered columnar body 79 may be a member having an optical function for selectively removing infrared rays.

The wavelength of infrared rays for which transmission is limited may be 650 nm or more. Thus, when a color image is taken by a common image-taking element, a light receiving component in a sensitive region of the image-taking element for a wavelength longer than that of R (red) light will not be superimposed on image data, thereby making it possible to prevent occurrence of color mixture.

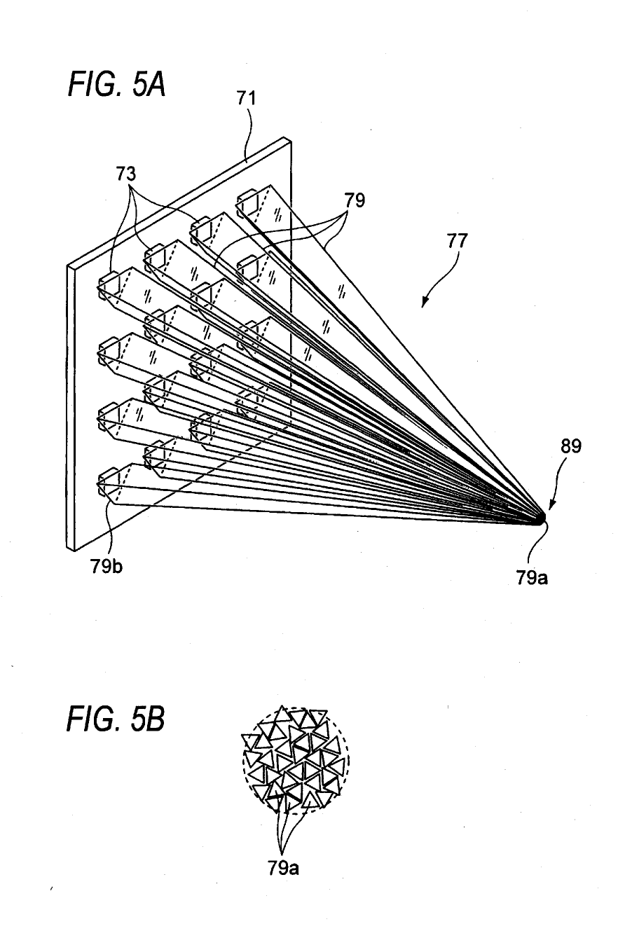

In the structure of the light source module 63 according to the present example, the white LEDs 73 are arranged in a 4 by 4 matrix on the support body 71 as illustrated in a light collecting member placement example provided in FIG. 5A. With the base end portions 79b of the tapered columnar bodies 79 opposed to the light emission surfaces of the respective white LEDs 73, the base end portions 79b are fixed via a transparent adhesive and/or an unillustrated fixing jig, for example. Furthermore, the tip portions 79a of the plurality of tapered columnar bodies 79 are bundled without being out of alignment thereof, thus forming the light emission window 89 with a minute size. When the light emission window 89 is partially enlarged, the tip portions 79a of the tapered columnar bodies 79 are bound together at a high density as illustrated in FIG. 5B. The respective tip portions 79a constitute the light emission window 89.

In this embodiment, as the foregoing white LEDs 73, surface mounting device (SMD) type LEDs or chip-on-board (COB) type LEDs directly implemented on the support body are used, and the light emission surface of each white LED 73 has an approximately square shape, the size of which is about 0.6 mm² to about 10 mm² and preferably about 1 mm². On the other hand, the area of the light emission window, formed by the tip portions 79a of the tapered columnar bodies 79, is 1 mm² to 5 mm² and preferably about 2 mm², and the total longitudinal length of each tapered columnar body 79 is about 20 mm.

In the light source device 47 with the above-described structure, the light emitted from the plurality of white LEDs 73 is introduced into the base end portions 79b of the tapered columnar bodies 79, guided by total reflection through the tapered columnar bodies 79, and then emitted as a high density light flux from the tip portions 79a. Accordingly, high intensity light is emitted with high efficiency through the light emission window 89 in which the tip portions 79a of the plurality of tapered columnar bodies 79 are bound together. As described above, the light emission window 89 is provided by optical connection of a large number of the tapered columnar bodies 79; hence, as seen from the light emission window 89, it looks as if an infinite number of illuminants are arranged in a distributed manner due to a large number of specular surfaces like a kaleidoscope. Consequently, the light emitted from the respective illuminants will not be scattered outside, and most of components of the emitted light are collected into the light emission window 89 to provide a high density light flux.

Moreover, the tip portions 79a of the respective tapered columnar bodies 79 are bound together while the positioning of the white LEDs 73 is maintained as it is, and therefore, the light may be collected into the light emission window 89 with a light emission pattern responsive to the amounts of light emitted from the respective white LEDs 73 and conforming to the alignment of the white LEDs 73 on the support body 71.

Note that the intensity of light emitted through the light emission window 89 has a distribution in which the intensity tends to be maximized at a center of the light emission window 89 and tends to decrease at a surrounding region located away from the center. Hence, even when the diameter of the light incidence surface of the light guide LG (which is equivalent to the diameter of the glass window 87 illustrated in FIG. 3) is changed in accordance with the type of the endoscope connected to the light source device 47, most of the emitted light will be introduced into the light incidence surface of the light guide LG and will not be leaked to the sleeve 81.

Accordingly, the light source module 63 and the light guide LG will be unaffected by heat-induced influences such as: temperature rises in the support body 71 and white LEDs 73 of the light emission part 75, which are caused by high intensity light applied to the sleeve 81 and reflected and returned to the light source; and a temperature rise in the light guide LG, which is caused by generation of heat by the sleeve 81 due to the light applied to the sleeve 81. Hence, even when different types of endoscopes, such as a nasotracheal endoscope, a peroral endoscope, a lower digestive system endoscope and a bronchoscope, are connected to the light source device 47, high intensity illumination light may be reliably applied to the inside of the light incidence surface of the light guide LG for each endoscope, and thus may be prevented from being applied to a region other than the light incidence surface, e.g., a surrounding region such as the sleeve 81.

Further, the foregoing support body 71 is not limited to a flat-plate support body, but may be a concave support body 71A such as one illustrated in FIG. 6. When the white LEDs 73 are arranged on a surface of the support body 71A in which a region thereof adjacent to the light collecting member 77 is formed into a concave shape, distances between the white LEDs 73 and the light emission window 89 may be uniformized irrespective of the positioning of the white LEDs 73, and the total lengths of the tapered columar bodies 79 may be aligned so as to be shortened. As a result, light emitted from the respective white LEDs 73 reach the light emission window 89 under the same conditions, thus eliminating a light amount difference resulting from a difference in the positioning of the white LEDs 73 on the support body 71. Besides, the tip portions 79a of the respective tapered columnar bodies 79 may be easily bundled while the positioning of the white LEDs 73 is maintained as it is.

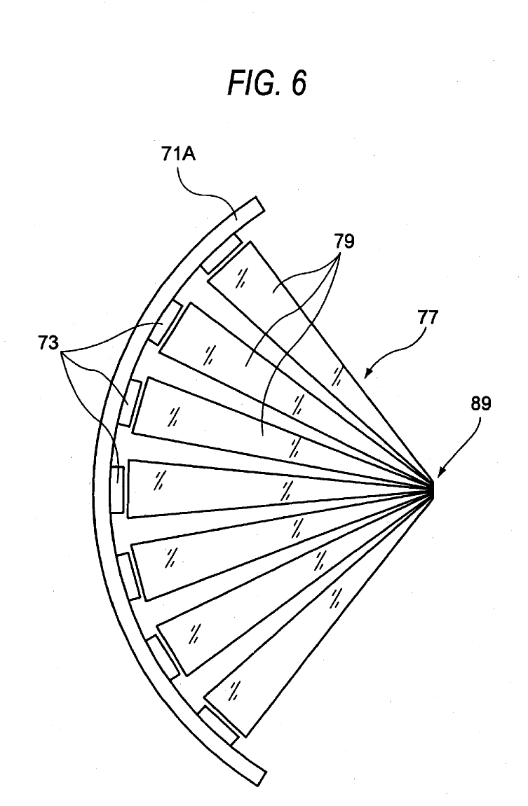

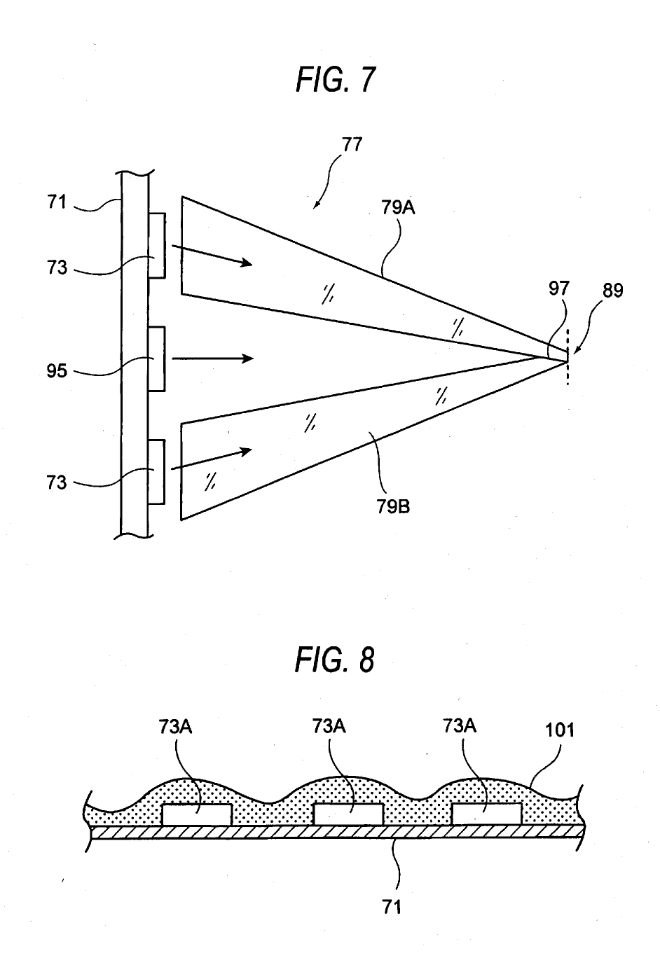

The relationship between the tapered columnar bodies 79 and the white LEDs 73 may be as follows. Each white LED 73 is provided for the associated one of the tapered columnar bodies; in addition, as illustrated in FIG. 7, a white LED 95 serving as an auxiliary illuminant may be located between tapered columnar bodies 79A and 79B, which are adjacent to each other, so as to emit light toward the light emission window 89. Light emitted from the white LED 95 in that case is emitted through a gap that is formed when the tip portions 79a of the tapered columnar bodies 79 are bundled as illustrated in FIG. 5B, and the amount of light emitted through the light emission window 89 is thus further increased.

Furthermore, when the tapered columnar body 79A is connected to the adjacent tapered columnar body 79B via a connection surface 97 as illustrated in FIG. 7, light from a plurality of the white LEDs 73 may be emitted in a combined manner through a tip portion of the single tapered columnar body 79A. Thus, an area of the light emission window 89, occupied by each illuminant, may be reduced, and the number of the tapered columnar bodies 79 bundled into the light emission window 89 may be increased. Hence, illumination light having a higher intensity may be produced by increasing the number of the illuminants that contribute to the production of the illumination light. Naturally, even when the illumination light having a higher intensity is produced, temperature rises in the light emission part 75 and the light guide LG may be more reliably prevented with the above-described structure.

Next, another mode of the light emission part 75 will be described below.

FIG. 8 is a schematic cross-sectional view of a light emission part in which a fluorescent layer is formed over the support body on which LEDs are implemented. In this structure, a plurality of blue LEDs 73A are arranged on the support body 71, and a fluorescent layer 101 including the foregoing fluorescent material is formed over the support body 71 and surfaces of the blue LEDs 73A. The fluorescent layer 101 is formed as follows. A liquid in which the fluorescent material is dispersed in a binding agent (binder) is applied, and is then dried and solidified, thereby forming the fluorescent layer 101.

Temperature rises in the support body 71 and the blue LEDs 73A may be prevented by forming the fluorescent layer 101 over the entire surface of the support body 71 as described above because even if reflection light is returned from the light emission window 89 located at the tip of the tapered columnar body 79, the reflection light is blocked by the fluorescent layer 101. Further, light is uniformly emitted from the entire support body 71 due to light emission of the blue LEDs 73A, thus also obtaining the effect of making it difficult to cause light amount variations in the light emission window 89.

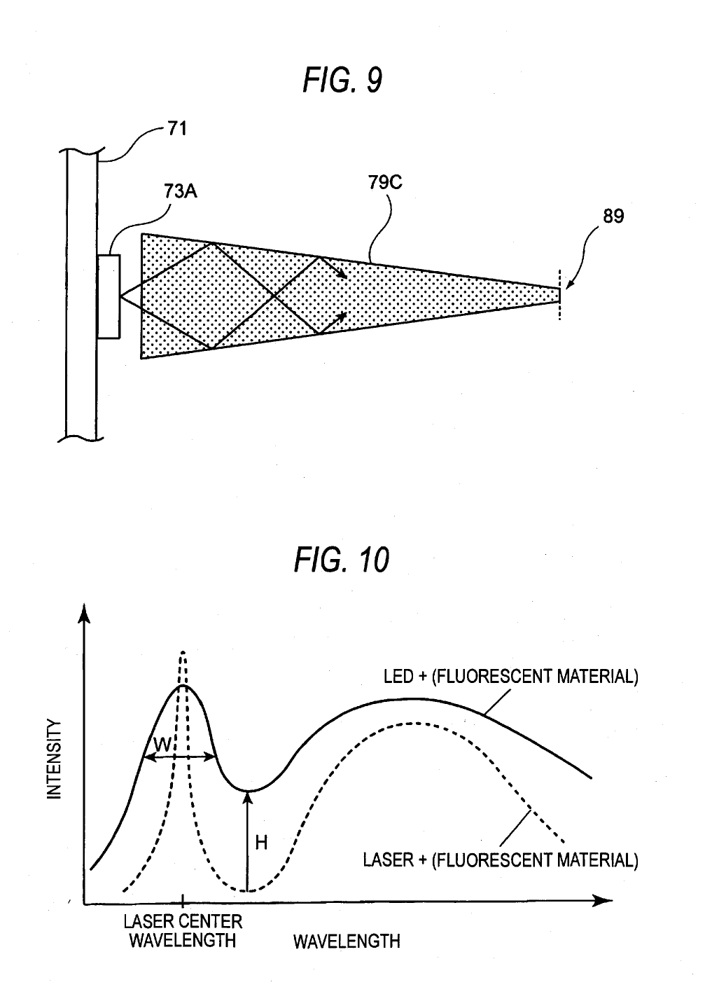

Furthermore, as illustrated in FIG. 9, the fluorescent material may be dispersed in a tapered columnar body 79C. In such a case, the fluorescent material is excited and emits light in the course of total reflection and guiding of light emitted from the blue LEDs 73A through the tapered columnar body 79C; then, most of components of the light emitted from the fluorescent material reach the light emission window 89, and are emitted therethrough. As a result, the components of the light emitted from the fluorescent material may be efficiently derived, which may contribute to an increase in emitted light amount.

Moreover, white light is produced by a combination of the light emitted from the LEDs and the light emitted from the fluorescent material as mentioned above, thereby making it possible to improve color rendering properties as compared with those obtained by white light produced by a combination of laser light and light emitted from the fluorescent material. In other words, as illustrated in one example of a light emission spectrum provided in FIG. 10, when white light is produced by a combination of laser light and light emitted from the fluorescent material, the wavelength range of short-wavelength laser light is narrow as indicated by the dotted line in FIG. 10, and a wavelength loss is likely to occur between a spectrum of laser light and that of fluorescent light from the fluorescent material.

On the other hand, when LEDs are used, a light emission spectrum width W of the LEDs is wider than that of laser light, and a spectrum of fluorescent light from the fluorescent material also provides light of a broad wavelength since light of various wavelength ranges makes contributions as excitation light. Besides, a wavelength loss is alleviated by an intensity increase H due to a wavelength component between the light emission of the LEDs and that of the fluorescent material. As a result, the white light, produced by a combination of the light emitted from the LEDs and the light emitted from the fluorescent material, has high color rendering properties, and thus serves as illumination light that is more suitable for observation.

The following description will be made on a structure example in which an infrared component is removed from light emitted from the light emission part 75 and then the light is introduced into the light guide LG in order to prevent heat generation at a connection between the light source module 63 and the light guide LG.

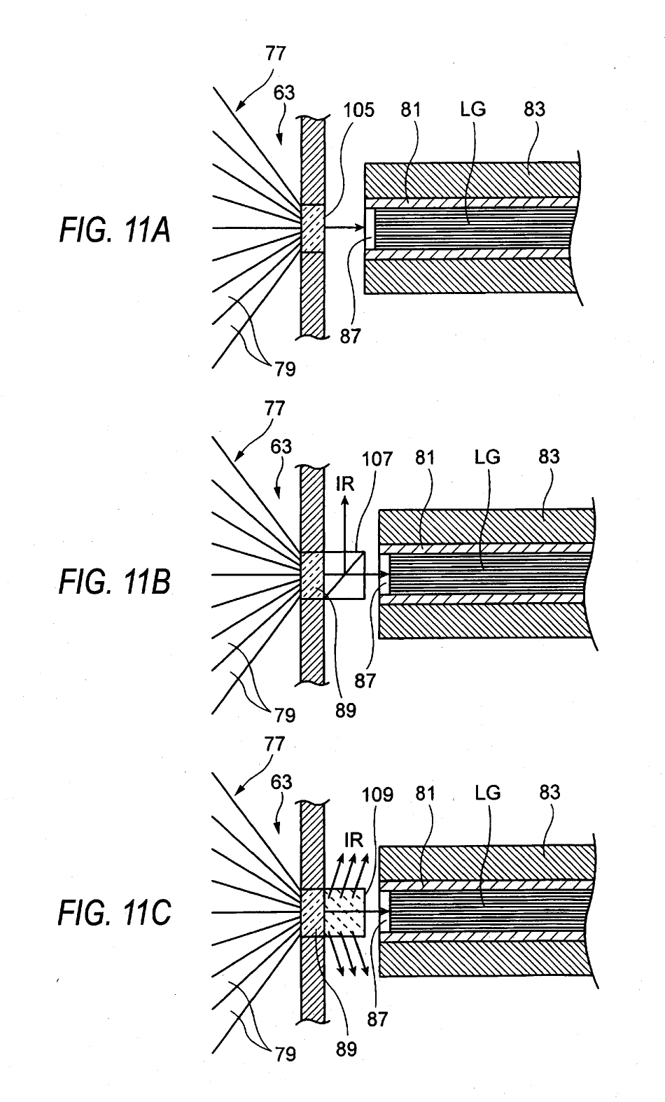

FIG. 11A is a schematic diagram illustrating an example in which an infrared absorber is provided as the light emission window of the light collecting member 77 including a plurality of the tapered columnar bodies 79. In the present structure example, an infrared cut filter 105 serving as an infrared absorber is provided between the light collecting member 77 and the light guide LG, and through this infrared cut filter 105, infrared rays (heat rays) are removed from light collected by the light collecting member 77, thus introducing only light components, transmitted through the infrared cut filter 105, into the light guide LG. As a result, a temperature rise resulting from light introduction is prevented at the light guide LG.

Further, although not illustrated, a reflection preventing film (AR coat layer) is formed at a surface of the infrared cut filter 105, thereby making it possible to eliminate reflection at an interface of the infrared cut filter 105, and to prevent generation of light returned to the light source.

FIG. 11B is a schematic diagram illustrating an example in which a stub having a multilayer reflection film for selectively reflecting an infrared component is provided at the light emission window 89 of the light collecting member 77. In the present structure example, a dichroic prism 107 having a multilayer reflection film is provided between the light collecting member 77 and the light guide LG, and through the dichroic prism 107, infrared rays IR are removed from light collected by the light collecting member, thereby introducing only light components, transmitted through the dichroic prism 107, into the light guide LG. As a result, a temperature rise at the light guide LG is prevented similarly to the foregoing example of FIG. 11A. Furthermore, the light emission window 89 formed of transparent glass is replaced with the infrared cut filter illustrated in FIG. 11A, thereby making it possible to more reliably remove infrared components. Note that similar effects are obtained also when a dichroic mirror is provided instead of the dichroic prism 107.

FIG. 11C is a schematic diagram illustrating an example in which a stub having an infrared reflection function is provided instead of the dichroic prism of FIG. 11B. In the present structure example, infrared reflection glass 109 is provided between the light collecting member 77 and the light guide LG. The infrared reflection glass 109 is provided by forming, at a surface of a transparent glass body, for example, a multilayered structure having titanium oxide and silicon oxide as main materials. As a result, a temperature rise at the light guide LG is prevented similarly to the foregoing examples of FIGS. 11A and 11B.

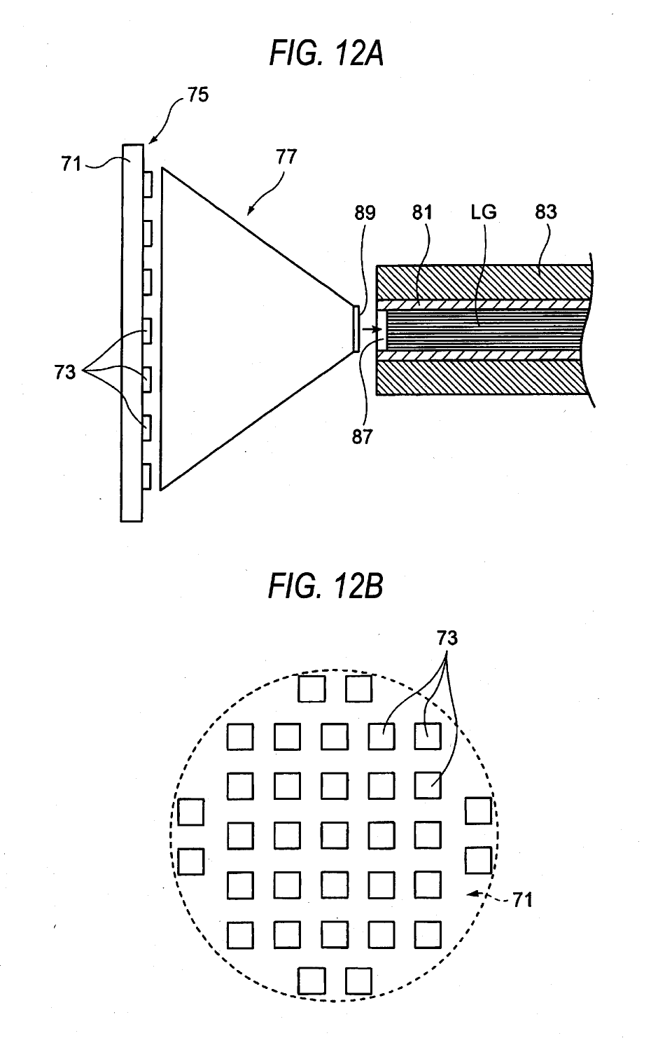

The following description will be made on exemplary control of the light emission part 75 for changing an application range of light, which is to be introduced into the light guide LG, by controlling the amount of light emitted from a plurality of the LEDs. FIG. 12A is an explanatory diagram schematically illustrating how light is introduced into the light guide LG when the light guide LG having a large diameter is connected to the light source device 47 (see FIG. 3). And FIG. 12B is a plan view illustrating the lit LEDs on the support body illustrated in FIG. 12A. Note that the number of the LEDs is 33 in the example illustrated in FIGS. 12A and 12B, but the number of the LEDs is not limited to this.

As illustrated in FIGS. 12A and 12B, the light emitted from the plurality of white LEDs 73 on the support body 71 is collected by the light collecting member 77 into a region located within the range of the light emission window 89, and is then introduced into the light guide LG. In the light collecting member 77, the above-mentioned tapered columnar bodies 79 are bundled without being out of the alignment thereof, and the arrangement pattern of the plurality of white LEDs 73 aligned on the support body 71 is thus reproduced as it is in the light emission window 89 in a scaled-down manner.

In this case, upon lighting of all the white LEDs 73 arranged on the support body 71, light is emitted from the entire range of the arrangement pattern from its center to its outer periphery, and the light is emitted toward the light guide LG through the entire light emission window 89.

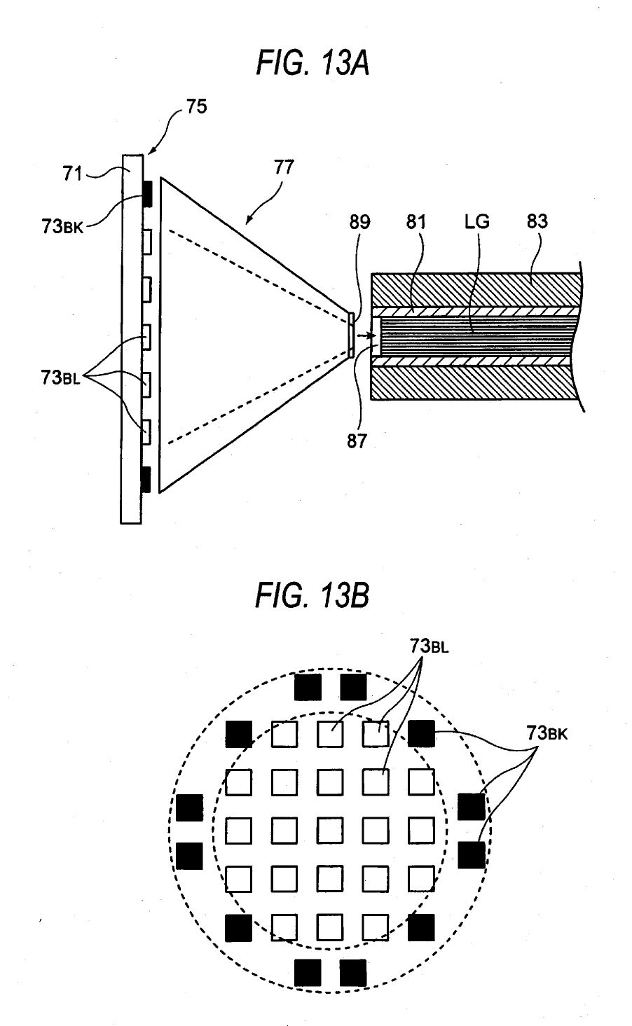

FIG. 13A is an explanatory diagram schematically illustrating how light is introduced into the light guide LG when the light guide LG having a small diameter is connected to the light source device 47 (see FIG. 3). And FIG. 13B is a plan view illustrating the lit LEDs on the support body illustrated in FIG. 13A.

As illustrated in FIGS. 13A and 13B, when an endoscope (such as a nasotracheal endoscope or a bronchoscope, for example) of a type different from that illustrated in FIGS. 12A and 12B is connected to the light source device 47, the diameter of the light guide LG is small. In this case, supposing that the plurality of white LEDs 73 arranged on the support body 71 include: white LEDs 73BK located close to an outermost edge of the support body 71; and white LEDs 73BL located on a center portion of the support body 71, power supplied to the white LEDs 73 is controlled so that power supplied to the white LEDs 73BK is shut off or reduced, and power supplied to the white LEDs 73BL is kept at a normal level or increased.

Then, the outer edge of light emitted from the white LEDs 73BL at the center portion is narrowed within a center range indicated by the dotted lines in FIG. 13A, and emission of light from an outer edge of the light emission window 89 is suppressed. As a result, even when the small diameter light guide LG is used, light is concentratedly introduced into the light incidence surface of the light guide LG, and therefore, no light will leak to a region other than the light incidence surface of the light guide LG, e.g., the sleeve 81.

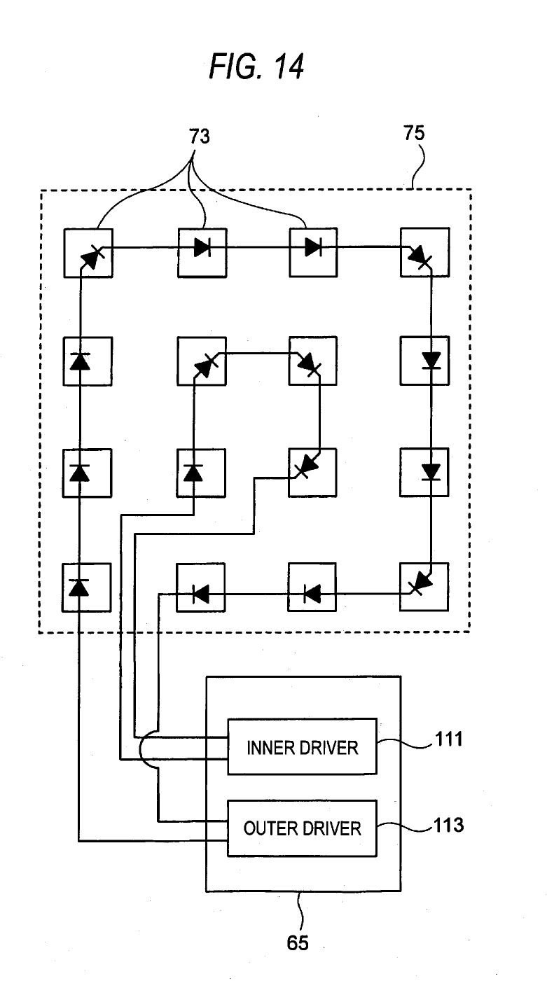

When the amounts of light emitted from a plurality of illuminants are selectively controlled as illustrated in FIGS. 13A and 13B, the light emission part 75 may have a connection structure such as one illustrated in FIG. 14. FIG. 14 illustrates a connection circuit of the light emission part 75 in a simplified manner on the assumption that the light emission part 75 has a structure in which the white LEDs 73 are arranged in a 4 by 4 matrix.

As illustrated in FIG. 14, the plurality of white LEDs 73 are arranged in a grid-like pattern and divided into inner and outer LED groups so that the inner and outer LED groups are controlled by an inner driver 111 and an outer driver 113, respectively. Although the LEDs are divided into two groups, i.e., inner and outer LED groups, in the example illustrated in FIG. 14, the number of groups into which the LEDs are divided may be further increased in the connection structure in accordance with the number of the illuminants. In such a case, the emitted light pattern may be controlled more minutely.

For example, when the endoscope 11 is connected to the light source device 47 as illustrated in FIG. 1, the control module 45 reads individual information stored in the memory 43 of the endoscope 11, and controls the light source driver 65 based on: the type of the connected endoscope 11 (including information concerning the diameter of the light guide LG); and information on various characteristics. In accordance with the diameter of the light guide LG of the connected endoscope 11, the light source driver 65 controls the amounts of light, emitted from the inner and outer LED groups illustrated in FIG. 14, with the use of the inner and outer drivers 111 and 113.

Specifically, when the large diameter light guide LG is used, the inner and outer LED groups are set at the same light amount; on the other hand, when the small diameter light guide LG is used, the light amount of the inner LED group is increased, and the light amount of the outer LED group is reduced or controlled so as to be extinguished. For light amount control, driving signal PWM control, pulse number control, pulse amplitude control or a combination thereof may be carried out in addition to current control, voltage control and ON/OFF control.

As described above, in the present structure example, illumination light may be emitted selectively within a suitable range corresponding to the type of the endoscope 11 connected to the light source device 47, and the light is prevented from being wastefully applied to a region other than the light guide LG. As a result, heat generation at a connection between the light source module 63 and the light guide LG may be prevented, and a temperature rise in the light source module 63, resulting from returned light, may be prevented.

Note that instead of performing light amount control for each LED group as illustrated in FIG. 14, a method of controlling light amount of each individual illuminant may be used for an illuminant connection circuit. In such a case, any pattern may be freely created as the pattern of light emitted through the light emission window 89.

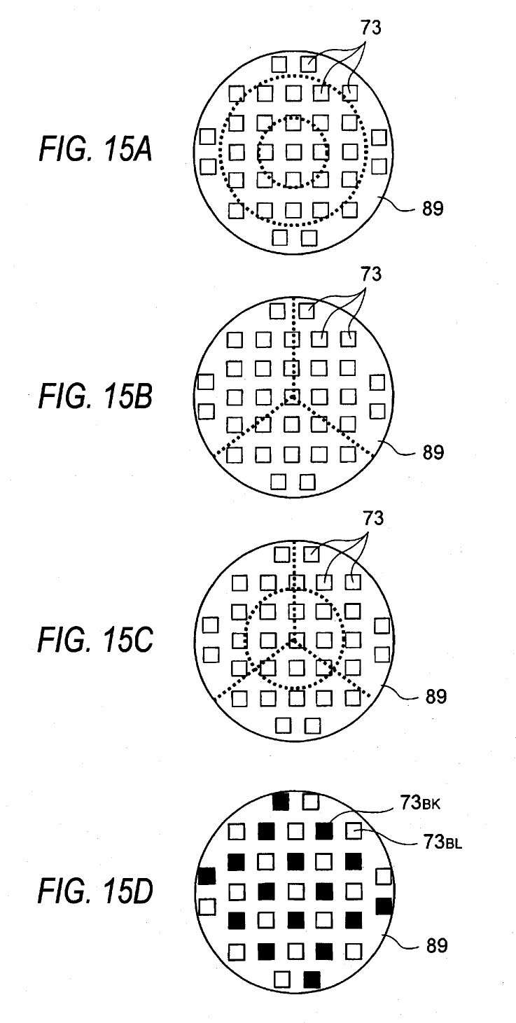

FIGS. 15A, 15B, 15C and 15D each schematically illustrate an example of an emitted light pattern in the light emission window 89. Each emitted light pattern is illustrated together with the positions of the white LEDs 73 in the diagram on the assumption that the arrangement pattern of the white LEDs 73 serving as illuminants is reproduced in the light emission window 89 as it is in a scaled-down manner.

FIG. 15A illustrates an example of an emitted light pattern in which the light emission window 89 is concentrically divided into blocks including a center block and outer annular blocks which are defined by the dotted lines in FIG. 15A. FIG. 15B illustrates an example of an emitted light pattern in which the light emission window 89 is circumferentially divided into blocks including a plurality of blocks defined at given circumferential angles. FIG. 15C illustrates an example in which radially divided blocks and circumferentially divided blocks are combined. And FIG. 15D illustrates an example in which light amounts are randomly set.

With these emitted light patterns, it is possible to perform adjustment in accordance with a difference in the diameter of the light guide LG, and in addition, it is also possible to perform adjustment for changing the amounts of light in a circumferential direction of the light emission window 89 with respect to its center, and to perform adjustment for uniformizing the amounts of light emitted through the entire light emission window 89.

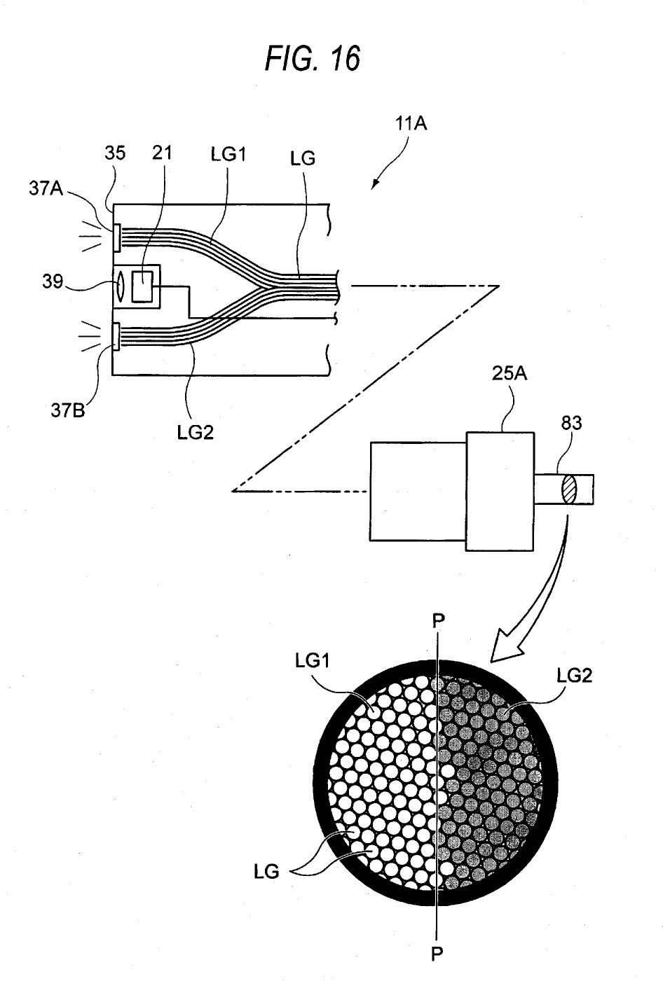

Specifically, when the light guide LG is placed as illustrated in FIG. 16, in which the tip portion 35 of an endoscope 11A and the connector 25A are provided, in such a manner that an image-taking optical system having the image-taking element 21 and the objective lens unit 39 at the endoscope tip portion 35 is sandwiched between light guides LG1 and LG2 branched out from the light guide LG, it is necessary to emit illumination light uniformly from both of application ports 37A and 37B connected to the light guides LG1 and LG2, respectively.

In the light guide LG contained in the protection pipe 83 protruded from the connector 25A, a bundle of the light guide LG1 and that of the light guide LG2 will not be usually mixed with each other, and the light guide LG is thus placed so as to be divided into the two light guides LG1 and LG2 along a boundary P-P. Therefore, when a light amount distribution exists in the circumferential direction of the light emission window 89, the amounts of light emitted from the application ports 37A and 37B become nonuniform.

In this case, the amounts of light emitted from the respective blocks are individually adjusted, thereby allowing uniform light amount to be supplied to the light guides LG1 and LG2, and allowing uniform illumination light to be emitted from both of the application ports 37A and 37B.

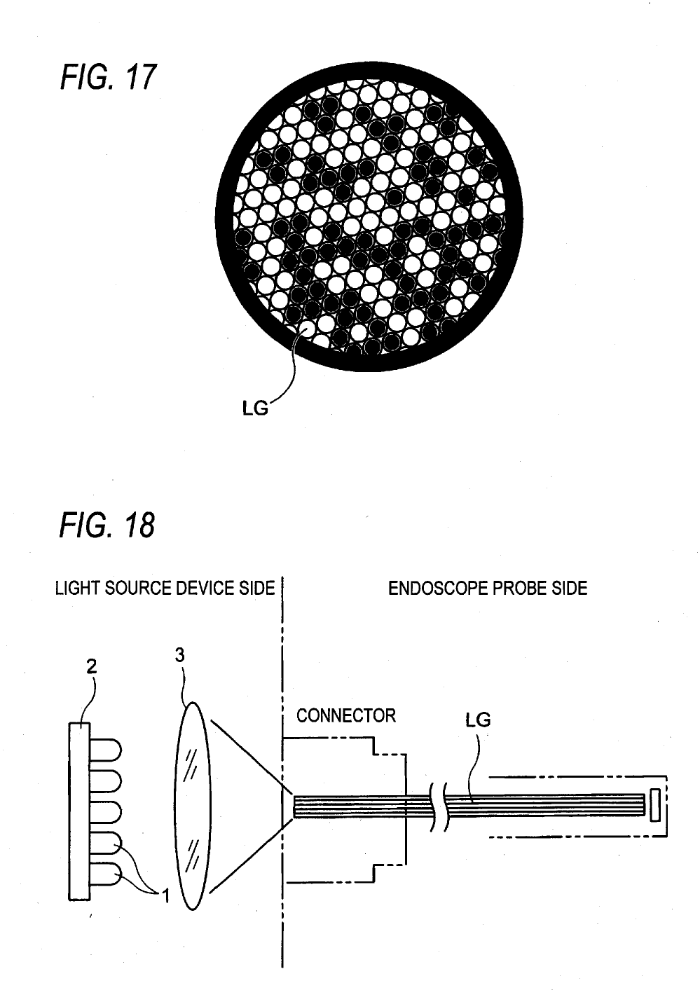

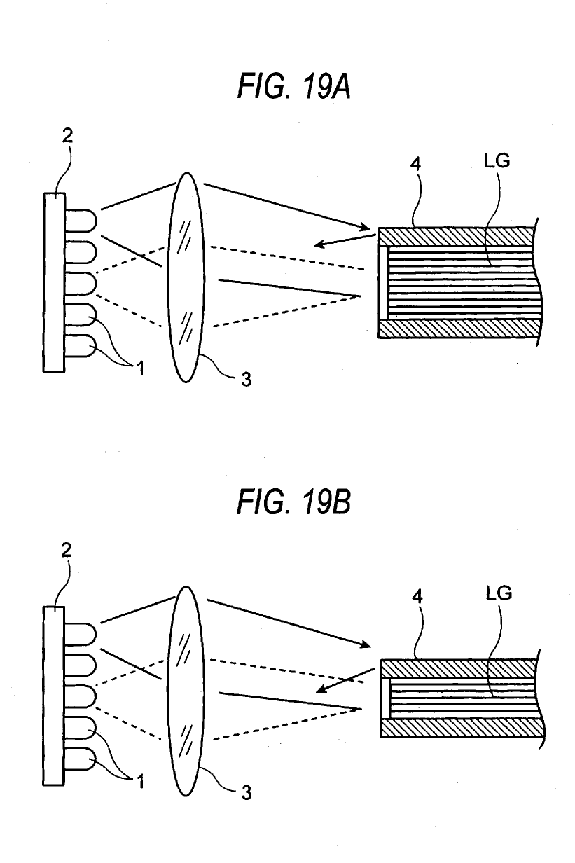

Moreover, in addition to individual control of emitted light amount for each block, skew processing for allowing optical fibers of the light guides LG1 and LG2 to be uniformly mixed with each other as illustrated in FIG. 17 may be performed. In that case, it is unnecessary to divide the light emission window 89 into blocks circumferentially.

Thus, the present invention is not limited to the foregoing embodiment, but it is intended that those skilled in the art may make changes or find applications of the present invention based on the description in the specification and known techniques, and such changes and applications fall within the scope of the protection. Specifically, although the example of application to a medical endoscope apparatus for observing and treating living body tissue has been provided in the foregoing description, the present invention is not limited to such application but may be applied to industrial endoscope apparatuses. Further, the present invention is not limited to endoscope apparatuses but may also be applicable to other lighting devices in which light is guided through fiber bundles. Furthermore, although LEDs are used as illuminants in the foregoing structure, the light source device may have an alternative structure in which laser light from a laser light source may be guided to each of light emission positions arranged in a grid pattern on the foregoing support body 71. Besides, the tapered columnar body 79 may be a tapered fiber that is formed by heating and drawing a multi-component glass fiber base material and that has a shape gradually reduced in diameter from its one end toward its other end. Moreover, light introduced into the tapered columnar body 79 does not necessarily have to be emitted from a single illuminant, but light emitted from a plurality of illuminants may be introduced into the tapered columnar body 79. In that case, the amounts of light emitted from the respective illuminants are individually controlled, thereby making it possible to increase a dynamic range of light intensity in the light emission window 89.

(1) According to an aspect of the invention, a light source device includes: a light emission part in which a plurality of illuminants are arranged on a support body; a light guide member in which light emitted from the light emission part is introduced into an incidence surface at one end of the light guide member and through which illumination light is emitted from an emission surface at the other end of the light guide member; and a light collecting member which is located between the light emission part and the light guide member and through which the light emitted from the light emission part is collected into the incidence surface of the light guide member, wherein the light collecting member includes a plurality of tapered columnar bodies tapered toward the light guide member from the light emission part, wherein the plurality of tapered columnar bodies are placed so that tip portions thereof are opposed to the incidence surface of the light guide member, and base end portions thereof are opposed to light emission surfaces of the illuminants, and wherein a selective translucent member that limits transmission of an infrared component, the selective translucent member being located at a portion along an optical path leading from the light emission part to the incidence surface of the light guide member.
(2) In the light source device of (1), the selective translucent member is an infrared absorber.
(3) In the light source device of (1), the selective translucent member comprises a multilayer reflection film for selectively reflecting at least an infrared component.
(4) In the light source device of (1), a reflection preventing film is formed at a surface of the selective translucent member.
(5) In the light source device of (1), the light guide member comprises: a large number of optical fiber bundles; and a sleeve made of zirconia ceramics that covers outer peripheries of the optical fiber bundles.
(6) In the light source device of (1), a fluorescent layer that emits light by being excited by the light from the illuminants is formed over the entire surface of the support body on which the illuminants are arranged.
(7) In the light source device of (1), the plurality of tapered columnar bodies are concentrically divided into a plurality of groups from a center of a light emission window, and for each of the plurality of groups, the amount of light emitted from the illuminants associated with the tapered columnar bodies are individually controlled by a light amount control unit.
(8) In the light source device of (7), the light amount control unit controls the amount of light emitted from the illuminants for each of the plurality of groups defined in a circumferential direction of the light emission window with respect to the incidence surface of the light guide member.
(9) In the light source device of (1), the illuminants are light emitting diodes.
(10) According to as aspect of the invention, an endoscope apparatus includes: the light source device of (1); and an endoscope that applies light, emitted from the light source device, to an observed region via the light guide member.
(11) In the light source device of (10), the endoscope comprises individual information retaining unit that retains individual information of the endoscope, and based on the individual information read from the individual information retaining unit, the light source device controls the amount of light emitted from the illuminants.

What is claimed is:

1. A light source device comprising:
a light emission part in which a plurality of illuminants are arranged on a support body;
a light guide member in which light emitted from the light emission part is introduced into an incidence surface at one end of the light guide member and through which illumination light is emitted from an emission surface at the other end of the light guide member; and
a light collecting member which is located between the light emission part and the light guide member and through which the light emitted from the light emission part is collected into the incidence surface of the light guide member,
wherein the light collecting member includes a plurality of tapered columnar bodies tapered toward the light guide member from the light emission part,
wherein the plurality of tapered columnar bodies are placed so that tip portions thereof are opposed to the incidence surface of the light guide member, and base end portions thereof are opposed to light emission surfaces of the illuminants, and
wherein a selective translucent member that limits transmission of an infrared component, the selective translucent member being located at a portion along an optical path leading from the light emission part to the incidence surface of the light guide member.

2. The light source device according to claim 1,
wherein the selective translucent member is an infrared absorber.

3. The light source device according to claim 1,
wherein the selective translucent member comprises a multilayer reflection film for selectively reflecting at least an infrared component.

4. The light source device according to claim 1,
wherein a reflection preventing film is formed at a surface of the selective translucent member.

5. The light source device according to claim 1,
wherein the light guide member comprises: a large number of optical fiber bundles; and a sleeve made of zirconia ceramics that covers outer peripheries of the optical fiber bundles.

6. The light source device according to claim 1,
wherein a fluorescent layer that emits light by being excited by the light from the illuminants is formed over the entire surface of the support body on which the illuminants are arranged.

7. The light source device according to claim 1,
wherein the plurality of tapered columnar bodies are concentrically divided into a plurality of groups from a center of a light emission window, and for each of the plurality of groups, the amount of light emitted from the illuminants associated with the tapered columnar bodies are individually controlled by a light amount control unit.

8. The light source device according to claim 7,
wherein the light amount control unit controls the amount of light emitted from the illuminants for each of the plurality of groups defined in a circumferential direction of the light emission window with respect to the incidence surface of the light guide member.

9. The light source device according to claim 1,
wherein the illuminants are light emitting diodes.

10. An endoscope apparatus comprising:
the light source device according to claim 1; and
an endoscope that applies light, emitted from the light source device, to an observed region via the light guide member.

11. The endoscope apparatus according to claim 10,
wherein the endoscope comprises individual information retaining unit that retains individual information of the endoscope, and
wherein based on the individual information read from the individual information retaining unit, the light source device controls the amount of light emitted from the illuminants.
